(19) United States
(12) Patent Application Publication
(10) Pub. No.: US 2023/0414685 A1
(43) Pub. Date: Dec. 28, 2023

(54) LACTOBACILLUS PLANTARUM PDA6 HAVING NEUROLOGICAL EFFECTS AND COMPOSITIONS INCLUDING THE SAME

(71) Applicant: Pouda Biotech LLC, Radnor, PA (US)

(72) Inventors: Jun ZHANG, Xian City (CN); Xin XIE, Xian City (CN)

(21) Appl. No.: 18/323,234

(22) Filed: May 24, 2023

Related U.S. Application Data
(60) Provisional application No. 63/347,279, filed on May 31, 2022.

(30) Foreign Application Priority Data
May 26, 2022 (CN) 202210585573.X

(51) Int. Cl. A61K 35/747; C12N 1/20; A61P 25/22

(52) U.S. Cl. CPC A61K 35/747; C12N 1/205; A61P 25/22; C12R 2001/25

(57) ABSTRACT

Provided are a Lactobacillus plantarum strain, Lactobacillus plantarum PDA6, and a composition thereof. Also provided are methods using Lactobacillus plantarum PDA6 or a composition thereof for treating anxiety disorder or depression.

### REFERENCE TO AN ELECTRONIC SEQUENCE LISTING

The contents of the electronic sequence listing 760288_401_SEQUENCE_LISTING.xml; Size: 3,271,698 bytes; and Date of Creation: May 19, 2023) is herein incorporated by reference in its entirety.

### TECHNICAL FIELD

The present disclosure relates to a Lactobacillus plantarum strain having neurological effects, Lactobacillus plantarum PDA6. Specifically, the present disclosure relates to a composition, including a pharmaceutical composition, which includes Lactobacillus plantarum PDA6 and a culture thereof. The present disclosure also relates to methods using Lactobacillus plantarum PDA6 or a composition containing Lactobacillus plantarum PDA6 to cause a neurological effect in a patient.

### BACKGROUND

The incidence of anxiety disorder and depression is increasing year by year. Unlike anxiety, which is a normal psychological response that can help people cope with the changing external environment, anxiety disorder is a pathological mental illness. Patients experiencing anxiety disorder often feel fear that is not tied to a specific external stimulus, or more intense or different type of fear for a longer duration than consistent with reality. For example, patients experiencing anxiety disorder may have an intense and disruptive fear of dying in the absence of any actual threat. Anxiety disorder often also produces a physiological response, such heart palpitation, chest tightness, dyspnea, nausea and vomiting, and sleep disorders, which can seriously endanger the patient's physical health and amplify or lead to other mental health disorders. See, e.g. Tully P J, et al. (2016). “Anxiety and Cardiovascular Disease Risk: a Review.” Curr Cardiol Rep 18(12): 120.

The pathogenesis of anxiety disorder is not fully understood such that there is no ideal target for drug discovery. Most drugs currently on the market or in clinical trials for the treatment of anxiety disorder exert an effect on nerve conduction and primarily target γ-aminobutyric acid (GABA) receptors or 5-hydroxytryptamine (5-HT) receptors. GABA and 5-HT are both important inhibitory neurotransmitters in the central nervous system of mammals (Wong C G, et al. (2003). “GABA, gamma-hydroxybutyric acid, and neurological disease.” Ann Neurol 54 Suppl 6: S3-12 and Deakin, J F W (2003). “Depression and Antisocial Personality Disorder: Two Contrasting Disorders of 5HT Function.” Springer, New York pages 79-93), and decreases in the concentration of these two neurotransmitters in the brain can cause people feel strong anxiety. Drugs, such as benzodiazepines and non-benzodiazepines, targeting GABA and receptors can reduce anxiety, presumably by mimicking the effects of GABA and 5-HT or by increasing their concentration in the brain. However, current drugs exhibit effects for only a short time after dosing and have many side effects, particularly with the type of long-term use required to treat anxiety disorder in most patients. These side effects include daytime fatigue, sleepiness, dry mouth, nausea, uncoordinated physical movements and other symptoms. In severe cases, patients may develop a dependency on anti-anxiety medications. Accordingly, use is often limited to short-term acute treatment of symptoms. Therefore, such drugs should not be the first-line medicines for clinical treatment of anxiety disorders or associated depression.

### SUMMARY

The present disclosure provides a novel Lactobacillus plantarum strain having neurological effects, Lactobacillus plantarum PDA6. In addition, the present disclosure provides a composition comprising Lactobacillus plantarum PDA6 and a culture thereof. The present disclosure further discloses methods using Lactobacillus plantarum PDA6 or a composition containing Lactobacillus plantarum PDA6 for treating anxiety disorder or depression.

In one aspect, the present disclosure provides an isolated Lactobacillus plantarum PDA6 strain deposited at Guangdong Microbial Culture Collection Center (GDMCC) under Accession No. 62262.

In another aspect, the present disclosure provides a composition comprising Lactobacillus plantarum PDA6 or a culture thereof and an excipient.

In another aspect, the present disclosure provides a method of treating anxiety disorder, comprising administering to a subject in need thereof a therapeutically effective amount of the isolated Lactobacillus plantarum PDA6 strain or a composition thereof.

In a further aspect, the present disclosure provides a method of treating depression, comprising administering to a subject in need thereof a therapeutically effective amount of the isolated Lactobacillus plantarum PDA6 strain or a composition thereof.

In a related aspect, the present disclosure provides an isolated Lactobacillus plantarum PDA6 strain or a composition thereof for use in a method of treating the human or animal body by therapy.

In another related aspect, the present disclosure provides an isolated Lactobacillus plantarum PDA6 strain or a composition thereof for use in a method of treating anxiety disorder or depression.

### BRIEF DESCRIPTION OF THE DRAWINGS

In order to more clearly describe the technical solutions in the embodiments of the present invention, the appended drawings used in embodiments are briefly described below.

FIG. 1 shows the treatment scheme for the mouse anxiety model as described in Example 2.

FIG. 2A-2D show the frequency of entering the central area and the duration of staying after entering the central area in the open field test as described in Example 3.

FIG. 3A-3D show the immobility time and the average immobility time in the water after the mice were forced to swim as described in Example 4.

FIG. 4A-4B show the changes in the content of BDNF and 5-HT as described in Example 5.

### DETAILED DESCRIPTION

Additional aspects and advantages of the present disclosure will become apparent to those skilled in this art from the following detailed description, wherein illustrative aspects of the present disclosure are shown and described. As will be appreciated, the present disclosure is capable of other and different aspects, and its several details are capable of modifications in various respects, all without departing from the disclosure. Accordingly, the descriptions are to be regarded as illustrative in nature, and not as restrictive.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The present disclosure provides a Lactobacillus plantarum strain having neurological effects, Lactobacillus plantarum PDA6. In addition, the present disclosure provides a composition, such as a pharmaceutical composition or a nutraceutical composition, which includes Lactobacillus plantarum PDA6 and a culture thereof. The present disclosure further discloses methods using Lactobacillus plantarum PDA6 or a composition containing Lactobacillus plantarum PDA6 for treating anxiety disorder or depression in a patient.

Lactobacillus plantarum PDA6 disclosed herein has one or more of the following characteristics:
a) acid resistance,
b) bile resistance,
c) no hemolytic activity against red blood cells,
d) weak antibiotic resistance,
e) capable of increasing expression of brain-derived neurotrophic factor (BDNF), 5-hydroxytryptamine (5-HT), and/or γ-aminobutyric acid (GABA) in the nervous system of a subject when administered to the subject,
f) capable of treating anxiety disorder,
g) capable of treating depression,
h) potentially fewer or less severe side effects (e.g., daytime fatigue, sleepiness, dry mouth, nausea, and/or uncoordinated physical movements) than conventional anti-anxiety disorder or depression drugs (e.g, benzodiazepines), and
h) useful as an active ingredient in a pharmaceutical or nutraceutical composition.
   Lactobacillus plantarum PDA6

In one aspect, the present disclosure provides a Lactobacillus plantarum strain, Lactobacillus plantarum PDA6.

As used herein, the term “Lactobacillus” refers to a microorganism belonging to the genus of aerobic or facultative anaerobic gram-positive bacillus widely distributed in nature. Microorganisms belonging to the genus Lactobacillus include Lactobacillus plantarum, etc.

The Lactobacillus plantarum PDA6 of the present disclosure was obtained by isolation from the intestinal tract of wasp. After isolation and purification, whole genomic sequencing confirmed that the strain is Lactobacillus plantarum, belonging to Firmicutes Lactobacillales, Lactiplantibacillus, Lactobacillus plantarum. The novel isolated Lactobacillus plantarum PDA6 was deposited at Guangdong Microbial Culture Collection Center (GDMCC), Institute of Microbiology, Guangdong Academy of Sciences, No. 59 Building, No. 100 Xianliezhong Road, Yuexiu District, Guangzhou 510075, China, under Accession No. 62262 on Mar. 4, 2022.

The term “isolated” refers to the state of a substance or microorganism separating from its naturally occurring environment. In certain embodiment, at least 50%, such as at least 60%, 70%, 80%, 90%, 95%, 97%, 98%, or 99%, of the materials in the naturally occurring environment other than the substance or microorganism of interest have been removed to generate an isolated substance or microorganism. In certain embodiments, an isolated substance or microorganism is at least 50%, such as at least 60%, 70%, 80%, 90%, 95%, 97%, 98%, or 99% pure. In other words, at least 50%, such as at least 60%, 70%, 80%, 90%, 95%, 97%, 98%, or 99% (by weight) of the isolated substance or microorganism is the substance or microorganism of interest.

The invention further provides a mutant, a variant, and/or a progeny of the deposited Lactobacillus plantarum PDA6 strain.

As used herein, the term “mutant” refers to any bacterium resulting from modification of the parent (i.e., deposited) strain. For example, a mutant may be a bacterium resulting from genetically modifying the deposited strain.

As used herein, the term “variant” refers to a naturally occurring bacterium derived from the parent (i.e., deposited) strain. For example, a variant may be a bacterium resulting from adaption to particular cell culture conditions.

As used herein, the term “progeny” means any bacterium resulting from the reproduction or multiplication of the deposited strain. Therefore, “progeny” means any direct descendant of the deposited strain. As such, the progeny strain may itself be identified as the same strain as the parent (i.e., deposited) strain. It will be apparent to one skilled in the art that due to the process of asexual reproduction, a progeny strain will be genetically virtually identical to the parent strain. Accordingly, in one embodiment, the progeny may be genetically identical to the parent strain and may be considered to be a “clone” of the parent strain. Alternatively, the progeny may be substantially genetically identical to the parent strain.

The mutant, variant or progeny may have at least 90, 95, 98, 99, 99.5 or 99.9% sequence identity over the entire length of the bacterial genome with their parent strain. Furthermore, the mutant, variant or progeny will retain the same phenotype as the deposited parent strain.

As used herein, “identical” or “identity” refers to the similarity between a DNA, RNA, nucleotide, amino acid, or protein sequence to another DNA, RNA, nucleotide, amino acid, or protein sequence. Identity can be expressed in terms of a percentage of sequence identity of a first sequence to a second sequence. Percent (%) sequence identity with respect to a reference DNA sequence can be the percentage of DNA nucleotides in a candidate sequence that are identical with the DNA nucleotides in the reference DNA sequence after aligning the sequences. Percent (%) sequence identity with respect to a reference amino acid sequence can be the percentage of amino acid residues in a candidate sequence that are identical with the amino acid residues in the reference amino acid sequence after aligning the sequences and introducing gaps, if necessary, to achieve the maximum percent sequence identity, and not considering any conservative substitutions as part of the sequence identity. The algorithm used herein for determining percent sequence identity is the BLAST 2.0 algorithm, as described in Altschul et al. “Gapped BLAST and PSI-BLAST: a new generation of protein database search programs,” Nucleic Acids Res. 2007, 25, 3389-3402, with the parameters set to default values.

As shown in Examples 3-5 below, the Lactobacillus plantarum PDA6 is capable of causing a neurological effect in an animal, such as alleviation of symptoms of anxiety disorder or depression. Without limiting the mechanism of action of the present disclosure, such effects may occur, at least in part, via producing GABA and/or as described in Mu, C., et al. (2016). “Gut Microbiota: The Brain Peacekeeper.” Front Microbiol 7: 345.

In some embodiments, the Lactobacillus plantarum PDA6 may cause fewer side effects or less intense side effects in a patient than an anti-anxiety disorder drug that targets a GABA or receptor, such as a benzodiazepine or non-benzodiazepine. Side effects include daytime fatigue, sleepiness, dry mouth, nausea, uncoordinated physical movements, or the like.

In some embodiments, the Lactobacillus plantarum PDA6 may be able to colonize the intestine of a patient, and may have resistance to acid and bile salts.

As used herein, the term “acid resistance” refers to the property of withstanding high acidity. If probiotics are acid-resistant, they can be prevented from being degraded or damaged even when exposed to strong acidic conditions in the stomach, by consumption through oral administration.

As used herein, the term “bile resistance” refers to the resistance to digestive enzymes in bile. Bile is made in the liver and stored in the gallbladder, and is a weak alkaline greenish-brown liquid that helps the digestion of fat in the duodenum of the small intestine, and contains various enzymes that help digestion and absorption by emulsifying fat. Bile is one of the major causes of reducing the effect of probiotic administration as it acts on probiotics ingested through oral administration.

The Lactobacillus plantarum PDA6 of the present disclosure may not exhibit a hemolytic activity against red blood cells. Hemolysis refers to the destruction of red blood cells and the release of hemoglobin to the surrounding area, and is an action by which the red blood cells are hemolyzed by enzymes produced from harmful bacteria in vivo. Therefore, the Lactobacillus plantarum PDA6 is recognized as a stable microorganism that does not cause hemolysis in the blood vessels even if it is administered in vivo.

In addition, the Lactobacillus plantarum PDA6 of the present disclosure may have a weak resistance to antibiotics. The antibiotics may specifically be, but are not limited to, ampicillin, clindamycin, gentamicin, kanamycin, erythromycin, ampicillin/sulbactam, chloramphenicol, or streptomycin. Accordingly, when the Lactobacillus plantarum PDA6 is used in pharmaceuticals, health functional foods, feed additives, etc., it has little or no resistance to antibiotics, and thus, the probability of causing related pharmacological effects or environmental problems is low.

Specifically, drug resistance genes in Lactobacillus plantarum PDA6 are provided in Table 1. Lactobacillus plantarum PDA6 may have weak resistance or no resistance to antibiotics or other drugs other than those connected with the resistance genes of Table 1.

TABLE 1
Lactobacillus plantarum PDA6 Drug Resistance
Genes (as identified by AMRFinderPlus, NCBI)

| Protein | Gene | Element | Element | | |
|---|---|---|---|---|---|
| Identifier | Symbol | Type | Subtype | Class | Subclass |
| SR1_g_00924 | ampC | AMR | AMR | BETA-LACTAM | BETA-LACTAM |
| SR1_g_01871 | blaOXA | AMR | AMR | BETA-LACTAM | BETA-LACTAM |
| SR1_g_03051 | Vat | AMR | AMR | STREPTOGRAMIN | STREPTOGRAMIN |
| SR1_g_04339 | merP | STRESS | METAL | MERCURY | MERCURY |
| SR1_g_04371 | merP | STRESS | METAL | MERCURY | MERCURY |
| SR1_g_04372 | merA | STRESS | METAL | MERCURY | MERCURY |
| SR1_g_04442 | silP | STRESS | METAL | SILVER | SILVER |
| SR1_g_04444 | SilA | STRESS | METAL | COPPER/SILVER | COPPER/SILVER |
| SR1_g_04445 | silB | STRESS | METAL | COPPER/SILVER | COPPER/SILVER |
| SR1_g_04446 | silF | STRESS | METAL | COPPER/SILVER | COPPER/SILVER |
| SR1_g_04447 | silC | STRESS | METAL | COPPER/SILVER | COPPER/SILVER |
| SR1_g_04448 | silR | STRESS | METAL | COPPER/SILVER | COPPER/SILVER |
| SR1_g_04449 | silS | STRESS | METAL | COPPER/SILVER | COPPER/SILVER |

In some embodiments, the Lactobacillus plantarum PDA6 bacteria may be modified using recombinant techniques to contain an antibiotic susceptibility gene or other gene that facilitates destruction of the bacteria after introduction into a patient.

In more specific embodiments, the Lactobacillus plantarum PDA6 may be provided as a probiotic.

In order to stably maintain the Lactobacillus plantarum PDA6 of the present disclosure for a long period of time, the strain may be stored by dissolving the cells in a storage solution prepared by mixing a certain amount of glycerol in water at −70° C., or may be freeze-dried by for example suspending the cells in sterilized 10% skim milk, but methods of maintaining the strain are not limited thereto.

Compositions Containing Lactobacillus plantarum PDA6

In another aspect of the present disclosure, there is provided a composition (e.g., a pharmaceutical composition or a nutraceutical composition) including the Lactobacillus plantarum PDA6 bacteria, a culture thereof, a concentrate thereof, or a dried form thereof and optionally an excipient.

In certain embodiments, the composition may comprise, consist essentially of, or consist of isolated Lactobacillus plantarum PDA6 bacteria. In other embodiments, the composition may be in a formulation for administration to a patient. In certain embodiments, the composition and/or the excipient is not naturally occurring.

The term “excipient” refers to an inactive substance that serves as the vehicle, carrier or medium for an active substance (e.g., Lactobacillus plantarum PDA6 bacteria or culture thereof). An excipient may be included in a composition for the purpose of long-term stabilization, bulking up solid formulations that contain potent active ingredients in small amounts, or to confer a therapeutic enhancement on the active ingredient in the final dosage form, such as facilitating drug absorption, reducing viscosity, or enhancing solubility. Excipients can also be useful in the manufacturing process, to aid in the handling of the active substance concerns such as by facilitating powder flowability or non-stick properties, in addition to aiding in vitro stability such as prevention of denaturation or aggregation over the expected shelf life.

Exemplary excipients including cryoprotectants (e.g., glycerol, trehalose, maltodextrin, skim milk powder, and starch), bulking agents, fillers, diluents, anti-adherents (e.g., magnesium stearate), binders (e.g., saccharides including sucrose, lactose, starches, cellulose, microcrystalline cellulose, hydroxypropyl cellulose, xylitol, sorbitol, or mannitol; proteins including gelatin; and synthetic polymers including polyvinylpyrrolidone (PVP) and polyethylene glycol (PEG)), coatings (e.g., hydroxypropyl methylcellulose, fatty acids, waxes, shellac, and plant fibers), disintegrants (e.g., crosslinked polymers including crosslinked polyvinylpyrrolidone, crosslinked sodium caboxymethyl cellulose, and sodium starch glycolate), flavoring agents (e.g., fruit extract), glidants (e.g., silica gel, fumed silica, talc and magnesium carbonate), lubricants (e.g., talc, silica, fats), preservatives (e.g., antixidants like vitamin A, vitamin E, vitamin C, retinyl palmitate and selenium; amino acids cysteine and methionine; citric acid and sodium citrate; and synthetic preservatives like the parabens (e.g., methyl paraben and propyl paraben), sorbents, sweeteners, and vehicles (e.g., dimethyl suloxide) for liquid and gel formulations.

In some embodiments, the composition is in the form of a liquid, a suspension, a dried (e.g., lyophilized) powder, a tablet, a capsule, a suppository, or an enema fluid, or a frozen liquid, suspension, or enema fluid. Dried compositions may be produced by air drying, natural drying, spray drying, freeze drying, lyophilization, and any combinations thereof. Compositions in the freeze dried state may include a protectant, such as a cryoprotectant, which may include ethylene glycol, dimethyl sulfoxide (DMSO), glycerol, and any combinations thereof. Such compositions may be reconstituted with a solution, such as a buffer, prior to oral, nasogastrical, or rectal administration.

In a liquid or suspension composition, the concentration of Lactobacillus plantarum PDA6 bacteria may be 10³ CFU/mL to 10⁸ CFU/mL, 10⁴ CFU/mL to 10⁸ CFU/mL, 10⁵ CFU/mL to 10⁸ CFU/mL, 10⁶ CFU/mL to 10⁸ CFU/mL, 10³ CFU/mL to 10⁷ CFU/mL, 10⁴ CFU/mL to 10⁷ CFU/mL, 10⁵ CFU/mL to 10⁷ CFU/mL, 10⁶ CFU/mL to 10⁷ CFU/mL, but is not limited thereto. In a solid composition, the amount of Lactobacillus plantarum PDA6 bacteria may be 10⁵ CFU/g to 10¹³ CFU/g, 10⁶ CFU/g to 10¹³ CFU/g, 10⁷ CFU/g to 10¹³ CFU/g, 10⁸ CFU/g to 10¹³ CFU/g, 10⁵ CFU/g to 10¹² CFU/g, 10⁶ CFU/g to 10¹² CFU/g, 10⁷ CFU/g to 10¹² CFU/g, 10⁸ CFU/g to 10¹² CFU/g, 10⁵ CFU/g to 10¹¹ CFU/g, 10⁶ CFU/g to 10¹¹ CFU/g, 10⁷ CFU/g to 10¹¹ CFU/g, 10⁸ CFU/g to 10¹¹ CFU/g, 10⁵ CFU/g to 10¹⁰ CFU/g, 10⁶ CFU/g to 10¹⁰ CFU/g, 10⁷ CFU/g to 10¹⁰ CFU/g, 10⁸ CFU/g to 10¹⁰ CFU/g, 10⁵ CFU/g to 10⁹ CFU/g, 10⁶ CFU/g to 10⁹ CFU/g, 10⁷ CFU/g to 10⁹ CFU/g, 10⁵ CFU/g to 10⁸ CFU/g, or 10⁶ CFU/g to 10⁸ CFU/g by weight of the composition.

In certain embodiments, the composition is provided in unit dosage form. The composition may comprise Lactobacillus plantarum PDA6 bacteria in an amount ranging 10⁵ CFU to 10¹³ CFU, 10⁶ CFU to 10¹³ CFU, 10⁷ CFU to 10¹³ CFU, 10⁸ CFU to 10¹³ CFU, 10⁵ CFU to 10¹² CFU, 10⁶ CFU to 10¹² CFU, 10⁷ CFU to 10¹² CFU, 10⁸ CFU to 10¹² CFU, 10⁵ CFU to 10¹¹ CFU, 10⁶ CFU to 10¹¹ CFU, 10⁷ CFU to 10¹¹ CFU, 10⁸ CFU to 10¹¹ CFU, 10⁵ CFU to 10¹⁰ CFU, 10⁶ CFU to 10¹⁰ CFU, 10⁷ CFU to 10¹⁰ CFU, 10⁸ CFU to 10¹⁰ CFU, 10⁵ CFU to 10⁹ CFU, 10⁶ CFU to 10⁹ CFU, 10⁷ CFU to 10⁹ CFU, 10⁵ CFU to 10⁸ CFU, or 10⁶ CFU to 10⁸ CFU per unit dosage form.

The composition provided herein preferably contains Lactobacillus plantarum PDA6 bacteria or culture thereof in an amount sufficient to colonize the intestine of a subject when administered to the subject.

In certain embodiments, the present disclosure provides a pharmaceutical composition comprising isolated Lactobacillus plantarum PDA6 bacteria and a pharmaceutically acceptable excipient.

A pharmaceutically acceptable excipient includes pharmaceutically acceptable cryoprotectants, bulking agents, fillers, diluents, anti-adherents, binders, coatings, disintegrants flavoring agents, glidants, lubricants, preservatives, sorbents, sweeteners, and vehicles or carriers.

In certain embodiments, the pharmaceutical composition includes at least one cryoprotectant selected from the group consisting of glycerol, trehalose, maltodextrin, skim milk powder, and starch. The cryoprotectant of the present disclosure may be contained in an amount of 0.01% to 20% by weight or 0.01% to 10% by weight based on the total weight of the composition. Specifically, based on the total weight of the composition, the glycerol may be contained in an amount of 5% to 20% by weight, the trehalose may be contained in an amount of 2% to 10% by weight, the maltodextrin may be contained in an amount of 2% to 10% by weight, the skim milk powder may be contained in an amount of 0.5% to 2% by weight, and the starch may be contained in an amount of 0.1% to 1% by weight in the composition.

In certain embodiments, the excipient may include at least one or any combination of diluent (Tris, citrate, acetate or phosphate buffers) having various pH values and ionic strengths, solubilizer such as Tween® or polysorbate, carriers such as human serum albumin or gelatin, preservatives such as thimerosal, parabens, benzylalconium chloride or benzyl alcohol, antioxidants such as ascorbic acid or sodium metabisulfite, and other components such as lysine, glycine, glucose, dextrin, skim milk, mannitol or succinylated β-lactoglobulin. In addition, the excipient may be contained in an amount of 50% to 95% by weight, 75% to 95% by weight or 85% to 95% by weight based on the total weight of the composition.

Selection of a particular pharmaceutical composition will depend upon a number of factors, including the condition being treated, the route of administration and the pharmacokinetic parameters desired. A more extensive survey of components suitable for pharmaceutical compositions is found in Remington's Pharmaceutical Sciences, 18th ed. A. R. Gennaro, ed. Mack, Easton, Pa. (1980).

The route of administration of a pharmaceutical composition eventually chosen will depend upon a number of factors and can be ascertained by one skilled in the art. In certain embodiments, the compositions can be further formulated using pharmaceutically acceptable carriers well known in the art in dosages suitable for oral, nasogastric, or rectal administration. Such carriers enable the pharmaceutical compositions to be formulated as tablets, pills, capsules, liquids, gels, syrups, slurries, suspensions, enema formulations, and the like, for oral, rectal or nasogastric administration to a patient to be treated. Formulations can include, for example, polyethylene glycol, cocoa butter, glycerol and the like.

In some embodiments, the pharmaceutical composition may further include one or more additional therapeutic agents, such as other anti-anxiety disorder or anti-depression agents.

The pharmaceutical composition can be administered in any sterile, biocompatible pharmaceutical carrier, including saline, buffered saline, dextrose, and water.

In certain embodiments, the present disclosure provides a nutraceutical composition comprising isolated Lactobacillus plantarum PDA6 bacteria or a culture thereof as an active ingredient. A “nutraceutical composition” refers to a food product or a food supplement capable of providing not only a nutritional effect and/or a taste satisfaction, but is also capable of delivering a therapeutic (or other beneficial) effect to a subject who consumes the composition. The nutraceutical composition may be in form of a probiotic composition, synbiotic composition, functional food, food supplement, or food product, including beverage.

The content of Lactobacillus plantarum PDA6 or cultures thereof in the nutraceutical composition may be based on the age or weight of the subject, desired effects, etc., and may be, for example, used in an amount of 0.0001% to 1% of the weight of the subject, such as 0.0001% to 0.001%, 0.0001% to 0.01%, 0.0001% to 0.1%, 0.001% to 0.01%, 0.001% to 0.1%, 0.01% to 0.01% to 1%, or 0.1 to 1% of the weight of the subject.

A nutraceutical composition of the present disclosure may further include a mixture of one or more of an organic acid such as citric acid, fumaric acid, adipic acid, lactic acid, etc.; a phosphate such as potassium phosphate, sodium phosphate, polyphosphate, etc.; or a natural antioxidant such as polyphenol, catechin, tocopherol, vitamin C, green tea extract, chitosan, tannic acid, etc.

In certain embodiments, isolated Lactobacillus plantarum PDA6, its culture, or a composition thereof may be used as a probiotic or probiotic composition. As used herein, the term “probiotics” refers to live bacteria that enter the body and provide a healthy benefit, such as improving the microbial balance of the body via, for example, ecological interactions with the resident flora in the digestive tract and/or reducing or eliminating symptoms or severity of a disorder (e.g., anxiety disorder or depression) via, for example, influencing the host physiology and immune system in a positive manner.

The probiotic composition provided herein may further comprise one or more other probiotics, such as other Lactobacillus, Bifidobacterium and Enterococcus in the form of fermented milk, granules, powder, etc.

In certain embodiments, the present disclosure provides a symbiotic composition comprising isolated Lactobacillus plantarum PDA6 or a culture thereof and one or more prebiotics. A “prebiotic” refers to a substance having a beneficial effect on a probiotic, such as food for Lactobacillus plantarum PDA6. Exemplary prebiotics include inulin, pectin and resistant starches.

In certain embodiments, the present disclosure provides a food supplement comprising isolated Lactobacillus plantarum PDA6 or a culture thereof and one or more prebiotics. As used herein, a “food supplement,” “supplement composition,” or “dietary supplement” refers to a composition that contains a dietary ingredient (e.g., isolated Lactobacillus plantarum PDA6 or a culture thereof) intended to add nutritional value or health benefits to supplement the diet. A food supplement may be added to a food, but that is not itself a food or intended to be a food, or that may be ingested independently of a food. The food supplement disclosed herein may comprise, in addition to isolated Lactobacillus plantarum PDA6 or a culture thereof, one or more dietary ingredients such as a vitamin, a mineral, a herb or other botanical, and an amino acid.

A food supplement of the present disclosure may be an immediate-release or sustained-release formulation, and may further include a carrier, such as an edible carrier. The edible carrier may include corn starch, lactose, sucrose, or propylene glycol. A solid carrier may be used for tablets, powders, troches, etc., and a liquid carrier may include syrups, liquid suspensions, emulsions, solutions, etc. Further, the food supplement may include a preservative, a lubricant, a solution accelerator, or a stabilizer.

The disclosure further provides a functional food that contains Lactobacillus plantarum PDA6 or a culture thereof. As used herein, the term “functional food” refers to a human food that has a beneficial effect (e.g., medical or physiological benefit other than a purely nutritional effect) in addition to providing nutrition.

In certain embodiments, the present disclosure provides a food product comprising isolated Lactobacillus plantarum PDA6 or a culture thereof and one or more prebiotics. A “food product” means a product or composition that is intended for consumption by a human or a non-human animal. Such food products include any food, feed, snack, food supplement, liquid, beverage, treat, toy (chewable and/or consumable toys), meal substitute or meal replacement.

In a functional food or a food product of the present disclosure, the Lactobacillus plantarum PDA6 or cultures thereof may be present in an amount of 0.0001% to 1% by weight, specifically 0.001% to 0.1% by weight, based on a raw material composition including the Lactobacillus plantarum PDA6. However, in the case of long-term administration, the amount may be less than the above-described range, such as 0.001% to 0.01% by weight based on a raw material composition including the Lactobacillus plantarum PDA6.

There is no particular limitation on the type of the food to which Lactobacillus plantarum PDA6 or cultures thereof may be included. Examples of foods to which Lactobacillus plantarum PDA6 or cultures thereof can be added include meats, sausages, breads, chocolates, candies, snacks, confectionaries, pizzas, instant noodles, other noodles, gums, dairy products including ice creams, yoghurt, curdled milk, and whole milk, various kinds of soup, beverages, teas, drinks, alcoholic drinks, vitamin complexes, etc., and all functional foods.

A beverage or drink comprising Lactobacillus plantarum PDA6 or cultures thereof may further contain various flavoring agents or natural carbohydrates, as in conventional drinks. The aforementioned natural carbohydrates may include monosaccharides such as glucose, fructose, etc.; disaccharides such as maltose, sucrose, etc.; polysaccharides such as dextrin, cyclodextrin, etc.; and sugar alcohols such as xylitol, sorbitol, erythritol, etc. Natural sweetening agents such as thaumatin, a stevia extract, etc.; and synthetic sweetening agents such as saccharin, aspartame, etc. may be used as the sweetening agent. A ratio of the additional components may be in a range of 0.01 to 0.04 parts by weight, specifically 0.02 to 0.03 parts by weight, based on 100 parts by weight of the Lactobacillus plantarum PDA6 or cultures thereof.

The present disclosure further includes a method of preparing a composition as described herein, which may include a step of mixing an additive with the Lactobacillus plantarum PDA6, a culture thereof, a concentrate thereof, or a dried form thereof. The additive may be the above-described excipient, such as a cryoprotectant, or a carrier.

The Lactobacillus plantarum PDA6 may be cultured by a conventional method for culturing Lactobacillus strains. As the medium, a natural medium or a synthetic medium may be used. As the carbon source of the medium, for example, glucose, sucrose, dextrin, glycerol, starch, etc. may be used. As the nitrogen source, peptone, meat extracts, yeast extracts, dried yeasts, soybean, ammonium salts, nitrate, and other organic or inorganic nitrogen-containing compounds may be used, but the nitrogen source is not limited thereto. As the inorganic salts included in the medium, magnesium, manganese, calcium, iron, potassium, etc. may be used, but the inorganic salts are not limited thereto. Amino acids, vitamins, nucleic acids, and related compounds may be added to the medium in addition to the carbon source, the nitrogen source, and the components of the inorganic salts. The Lactobacillus plantarum PDA6 may be cultured for 12 hours to 4 days in a temperature range of 20° C. to 40° C.

Specifically, the culture broth of the Lactobacillus plantarum PDA6 may be a crude culture broth containing cells, and may also be cells from which a culture supernatant is removed, or concentrated cells. The composition of the culture broth may additionally contain not only components required for conventional culture of Lactobacillus, but also components that act synergistically to the growth of Lactobacillus, and the compositions thereof may be readily selected by those skilled in the art.

Methods of Treatment Using Lactobacillus plantarum PDA6

In another aspect, the present disclosure provides a method for treating anxiety disorder comprising administering to a subject in need thereof a therapeutically effective amount of the isolated Lactobacillus plantarum PDA6 strain or a composition (e.g., a pharmaceutical composition) comprising the isolated Lactobacillus plantarum PDA6 strain or a culture thereof provided herein.

In a further aspect, the present disclosure provides a method of treating depression (e.g., anxiety disorder-associated depression), comprising administering to a subject in need thereof a therapeutically effective amount of the isolated Lactobacillus plantarum PDA6 strain or a composition (e.g., a pharmaceutical composition) comprising the isolated Lactobacillus plantarum PDA6 strain or a culture thereof provided herein.

In related aspects, the present disclosure provides the isolated Lactobacillus plantarum PDA6 strain or a composition (e.g., a pharmaceutical composition) comprising the isolated Lactobacillus plantarum PDA6 strain or a culture thereof as described above for use in a method of treating the human or animal body by therapy, such as in a method for treating anxiety disorder or depression.

As used herein, “anxiety disorder” may be any psychological disorder diagnosed as such according to applicable clinical guidelines. In some embodiments, anxiety disorder may be generalized anxiety disorder, panic disorder, social anxiety disorder, post traumatic stress disorder (PTSD), or any combinations thereof. In some embodiments, the anxiety disorder may be any “Anxiety Disorder” as defined in the Diagnostic and Statistical Manual of Mental Disorders, 5^{th} Edition (DSM-5-TR). In some embodiments, a symptom of anxiety disorder may include excessive anxiety and worry (apprehensive expectation), which optionally may occur more days than not for at least 6 months, and/or be about a number of events or activities (such as work or school performance); difficulty in controlling worry; restlessness or feeling keyed up or on edge; being easily fatigued; difficulty concentrating; mind going blank; irritability; muscle tension; sleep disturbance (e.g. difficulty falling or staying asleep, or restless unsatisfying sleep); clinically significant distress; clinically significant impairment in social, occupational, or other important areas of functioning, depression, or any combinations thereof, optionally not attributable to another psychological condition or medication.

As used herein, “depression” may be any psychological disorder diagnosed as such according to applicable clinical guidelines. Depression is a mood disorder that causes a persistent feeling of sadness and loss of interest, which affects how one feels, thinks and behaves and can lead to a variety of emotional and physical problems. Symptoms of depression include feelings of sadness, tearfulness, emptiness or hopelessness; angry outbursts, irritability or frustration even over small matters, loss of interest or pleasure in most or all normal activities; sleep disturbances, including insomnia or sleeping too much; tiredness and lack of energy; reduced appetite and weight loss or increased cravings for food and weight gain; anxiety, agitation or restlessness; slowed thinking, speaking or body movements; feelings of worthlessness or guilt, fixating on past failures or self-blame; trouble thinking, concentrating, making decisions and remembering things; frequent or recurrent thoughts of death, suicidal thoughts, suicide attempts or suicide; unexplained physical problems, such as back pain or headaches.

An “individual,” “subject,” or “patient” herein is a vertebrate, such as a human or non-human animal, for example, a mammal. Mammals include, but are not limited to, humans, non-primates, farm animals, sport animals, rodents and pets. Examples of non-human animal subjects include rodents such as mice, rats, hamsters, and guinea pigs; rabbits; dogs; cats; sheep; pigs; goats; cattle; horses; and non-human primates such as apes and monkeys.

A “therapeutically effective amount” of a substance as used herein is that amount sufficient to effect beneficial or desired results, including clinical results, and, as such, an “effective amount” depends upon the context in which it is being applied. In some embodiments, in the context of administering a composition to treat anxiety disorder, the severity, frequency, or duration of at least one symptom of anxiety disorder may be reduced when a therapeutically effective amount is administered.

As used herein, and as understood in the art, “treatment” or “treating” is an approach for obtaining beneficial or desired results, including clinical results. For purposes of this subject matter, beneficial or desired clinical results include, but are not limited to, prevention, alleviation or amelioration of one or more symptoms, diminishment of extent of disease, stabilized (i.e., not worsening) state of disease, prevention of disease, delay or slowing of disease progression, reduced risk of recurrence of disease, and/or amelioration or palliation of the disease state.

Lactobacillus plantarum PDA6, a composition, or a food or supplement composition as described herein may be administered to a subject. In particular, a therapeutically effective amount of the Lactobacillus plantarum PDA6, a composition, or a food or supplement composition as described herein may be administered to a subject having anxiety disorder to treat the anxiety disorder or to a subject having depression to treat depression. The therapeutically effective amount of the composition, or food or supplement composition may depend on many factors, such as the age, weight, and severity of anxiety disorder or depression in the subject, the length of treatment, other therapeutics concurrently administered to the subject, and gastrointestinal conditions of the subject.

In some embodiments, the Lactobacillus plantarum PDA6, a composition, or a food or supplement composition may be administered in an amount sufficient for detectable colonization of the intestine of the subject with Lactobacillus plantarum PDA6. Colonization may be tested a set period of time after administration of the Lactobacillus plantarum PDA6 or a composition comprising the Lactobacillus plantarum PDA6 bacteria or a culture thereof, such as after one day, one week, two weeks, three weeks, or one month.

In specific embodiments, Lactobacillus plantarum PDA6 or a composition thereof may be administered with another anti-anxiety disorder or anti-depression therapeutic. The administration of the other therapeutic may be at the same time as, prior to, or subsequent to the administration of Lactobacillus plantarum PDA6 or a composition thereof. Dosage of the other therapeutic may be reduced over time as the Lactobacillus plantarum PDA6 colonizes the intestine of the subject.

The Lactobacillus plantarum PDA6 or a composition thereof may be administered to a subject once, or multiple times, such as daily, weekly, biweekly, every three weeks, monthly, every two months, every six months, every year, or whenever the Lactobacillus plantarum PDA6 is not detectable in the intestine of the subject.

The subject may be a human, but, in some embodiments, it may be another animal susceptible to anxiety disorder or depression, such as a dog, cat, cow, goat, or horse.

The Lactobacillus plantarum PDA6 or a composition thereof may be administered to a subject orally, nasogastricly or rectally. Preferably, the administration is oral administration.

In some embodiments, the Lactobacillus plantarum PDA6 or a composition thereof may be administered so that the Lactobacillus plantarum PDA6 dose is an amount of 10⁵ CFU/day or more, 10⁶ CFU/day or more, 10⁷ CFU/day or more, 10⁸ CFU/day or more, 10⁹ CFU/day or more, 10¹⁰ CFU/day or more, 10¹¹ CFU/day or more, 10¹² CFU/day or more, or 10¹³ CFU/day or more, or in a range between any of these amounts (e.g., 10⁵ CFU to 10¹³ CFU per day).

The Lactobacillus plantarum PDA6 or a composition thereof may be administered repeatedly until at least one symptom of anxiety disorder or depression in the subject improves.

### EXAMPLES

The presently disclosed subject matter will be better understood by reference to the following Examples, which are provided as exemplary of the invention, and not by way of limitation.

### Example 1: Identification of Lactobacillus plantarum PDA6

The bacterial strain from the wasp intestine was separated and purified to form isolated Lactobacillus plantarum PDA6. A Biospin Bacteria Genomic DNA Extraction Kit was used to extract DNA from the isolated bacteria. Purity and concentration of DNA was determined. Covaris M220 was used to fragment genomic DNA, and an Invitrogen Qubit 4.0 fluorescence quantifier was used for concentration quantification. A KAPA Hyper Prep Kit was used for library construction and the Illumina NovaSeq was used for sequencing. 43 contigs (SEQ ID NOS: 1-43) were obtained.

The original sequences obtained were classified and the genome was assembled and compared with the 63,400 assembled gene sequences of NCBI by MASH (version: 2.2). Identification was based on mash distance information. Similarity to Lactobacillus plantarum was more than 99%. Therefore, the newly isolated strain was named Lactobacillus plantarum PDA6.

### Example 2: Establishment of Mouse Anxiety Model

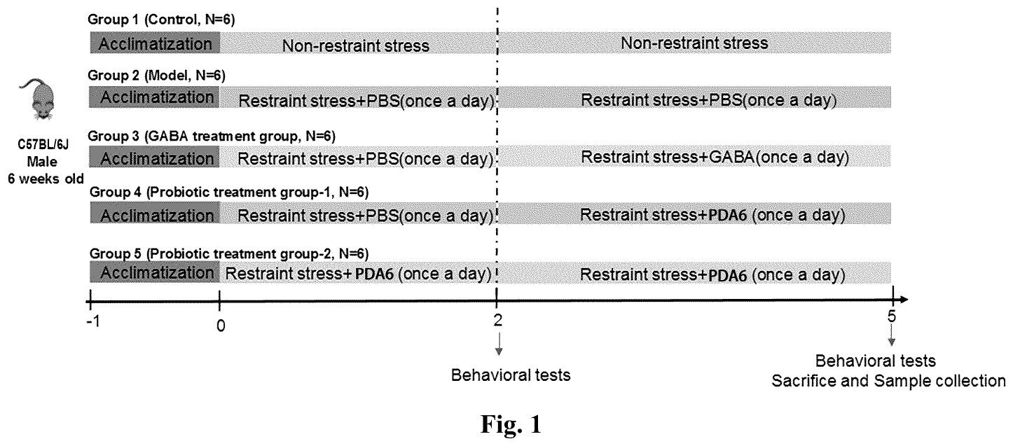

After a preliminary feeding, male C57BL/6J (6-week-old) mice were randomly divided into a normal control group (no restraint; no PBS or therapeutics), a model group (restraint; PBS once a day), a GABA drug treatment group (restraint; 0.5 mg/mL, 0.2 mL/Day, administration of GABA begun after 2 weeks), Lactobacillus plantarum group P1 (restraint; 1×10⁸ CFU/mL, gavage of Lactobacillus plantarum begun after 2 weeks), and Lactobacillus plantarum group P2 (restraint; 1×10⁸ CFU/mL, gavage of Lactobacillus plantarum begun immediately). Each group contained 6 mice. The mice in each group except the control group were under restraint stress once a day. The treatment scheme is shown in FIG. 1. Behavioral tests and related sample collection were performed at 2 weeks and 5 weeks, respectively, as set forth in Examples 3-5.

### Example 3: Open Field Test

An open field test was conducted using an experimental device including a 50 cm×50 cm×50 cm open box made of ABS engineering plastics. The bottom and inner wall of the box were white. The box was equipped with a camera for automatic recording. The camera was connected to a computer, and recorded visual field was divided into a central area and a peripheral area using behavioral software. The central area occupied 25% of the field. The software synchronously tracked and recognized the movement of the mouse. The software automatically recognized the mouse and whether it was moving or still and recorded frequency of entry into the central area and time spent in the central area. After each experiment, the bottom of the box was wiped with 75% alcohol to remove mouse excrement and residual odor.

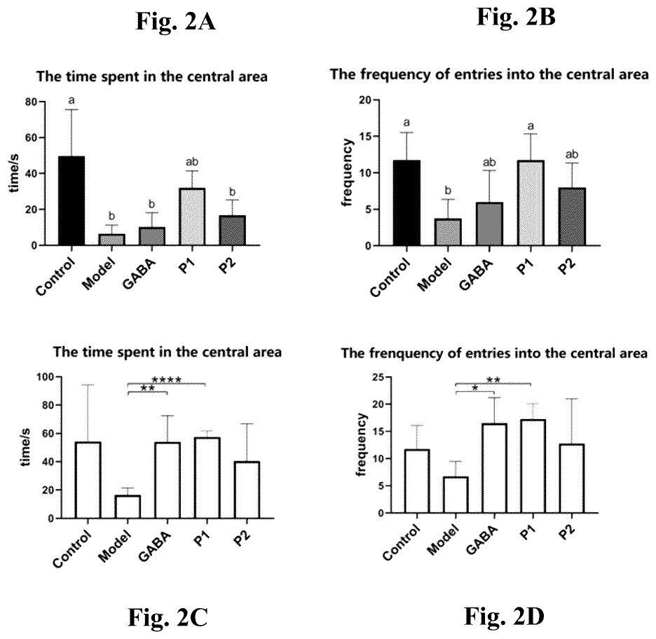

To conduct the test, a mouse was placed in the central area and allowed to adapt for 1 minute before a timer was started. The mouse was allowed to move freely for 5 minutes while the movement was recorded. Results are shown in FIG. 2.

FIG. 2, parts A and B show the results for each group after two weeks. As compared to the model group mice, the time spent in the central area in the open field of the mice in the P1 and P2 groups was increased although the differences were not significant (FIG. 2, part A). Also compared to the model group mice, the frequency of entering the central area in the open field of the mice in the P1 and P2 groups was increased (FIG. 2, part B). While the increase in the P2 group was not statistically significant, the increase in the P1 group was significant. The lack of significant differences between the P1 and the model group in the time spent in the central area and between the P2 group and the model group in both the time spent in the central area and the frequency of entering the central area may be due to the Lactobacillus plantarum having not yet colonized the intestines of the mice in the P1 and P2 groups.

FIG. 2, parts C and D show the results for each group after five weeks. The time spent in the central area and the frequency of entries of the mice in the GABA group and P1 group were significantly increased compared with the model group. There was also an increasing trend in the P2 group. Both of these indicators are negatively correlated with anxiety-like behaviors, suggesting that Lactobacillus plantarum induced a reduction in anxiety-like behaviors.

### Example 4: Forced Swimming Test

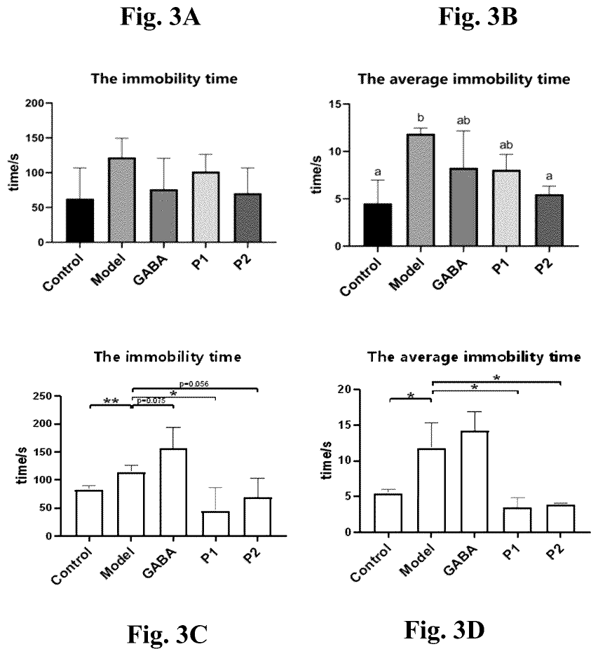

To conduce a forced swimming test, the mice were placed in a cylindrical glass tank with a height of 20 cm and a diameter of 10 cm, one mouse in each tank. The water depth in the tank was 10 cm, and the water temperature was (25±1) ° C. The mice in the training group were trained to swim for 15 min before the experiment, then were removed and put back into the cage, and the experiment was conducted 24 h later. During the experiment, the mice swam in the tank for 6 minutes, and the cumulative immobility time and the average immobility time of each mouse in the next 4 minutes was recorded. Immobility time is time during which the mouse stopped struggling in the water, floats, or only moved an amount needed to keep its head above water. The more anxious and depressed animals were, the longer they remained immobile in the water. Results are shown in FIG. 3.

FIG. 3, parts A and B show results after two weeks. Compared with the control group, the immobility time of the mice in the other groups was higher and the average immobility time of the mice in the model group was significantly increased. The average immobility time of the P2 group was significantly decreased compared with that of the model group.

FIG. 3, parts C and D show results after five weeks. Compared with the control group, the cumulative immobility time and the average resting time per time of the model group were significantly increased, while in each of the two groups that received Lactobacillus plantarum, the cumulative immobility time and the average resting time per time were significantly reduced compared with the model group. Interestingly, compared with the model group, the GABA group did not exhibit significantly reduced cumulative immobility time or average immobility time. These results suggested that Lactobacillus plantarum had a better therapeutic effect than GABA, which may be related to the use of other probiotic functions to positively regulate the behavior of mice in addition to probiotics that affects the depression symptoms by producing GABA.

### Example 5: Expression of BDNF and 5-HT in Hippocampus

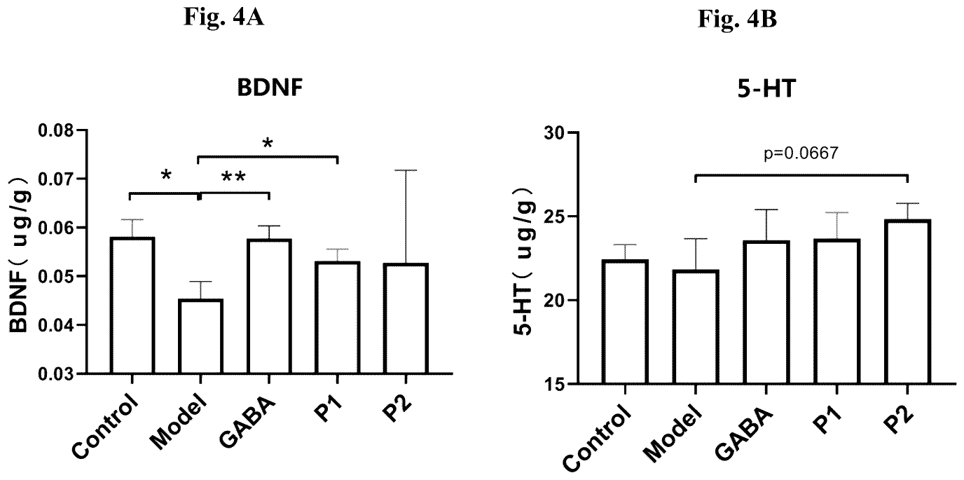

After the behavioral tests, the hippocampus tissues from the brain of the mouse were isolated, and pulped in normal saline, then centrifuged at 3000 rpm for 10 minutes. The supernatant was removed and used to conduct ELISAs for brain-derived neurotrophic factor (BDNF) and 5-HT. BDNF is a protein synthesized in the brain and widely distributed in the central nervous system that plays an important role in the survival, differentiation, growth and development of neurons. 5-HT is a monoamine neurotransmitter in the central nervous system and is associated with feelings of happiness. If a mouse has anxiety symptoms, the expression of these two substances in the brain is down-regulated. The ELISA results are shown in FIG. 4.

After restraint stress, the expression of BDNF in the hippocampus of the model mice was significantly reduced. After the administration of GABA and Lactobacillus plantarum, the expression of BDNF was significantly increased (FIG. 4, part A). As shown in FIG. 4, part B, compared with the model group, the 5-HT expression level in the P1 and P2 mice increased by about 0.5 times.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An isolated Lactobacillus plantarum PDA6 strain deposited at Guangdong Microbial Culture Collection Center (GDMCC) under Accession No. 62262.

2. A composition comprising the isolated Lactobacillus plantarum PDA6 strain of claim 1 or a culture thereof and an excipient.

3. The composition of claim 2, wherein the composition is not naturally occurring.

4. The composition of claim 2, wherein the composition is a pharmaceutical composition.

5. The composition of claim 4, wherein the composition is formulated for oral, nasogastric, or rectal administration.

6. The composition of claim 2, wherein the composition is a nutraceutical composition.

7. The composition of claim 6, wherein the composition is in form of a probiotic composition, synbiotic composition, food supplement, functional food, food product, or beverage.

8. The composition of claim 2, wherein the composition is a liquid.

9. The composition according to claim 2, wherein the composition is dried.

10. The composition according to claim 9, wherein the composition is lyophilized or freeze-dried.

11. The composition according to claim 2, comprising Lactobacillus plantarum PDA6 in an amount sufficient to colonize the intestine of a subject when administered to the subject.

12. The composition according to claim 2, comprising Lactobacillus plantarum PDA6 in an amount ranging from 10³ CFU/mL to 10⁸ CFU/mL or 10⁵ CFU/g to 10¹³ CFU/g.

13. The composition according to claim 2, wherein the composition is in unit dosage form.

14. The composition according to claim 13, wherein Lactobacillus plantarum PDA6 is present in the composition in an amount between 10⁵ CFU to 10¹³ CFU per unit dose.

15. A method of treating anxiety disorder, comprising administering to a subject in need thereof a therapeutically effective amount of the isolated Lactobacillus plantarum PDA6 strain according to claim 1.

16. The method of claim 15, wherein the therapeutically effective amount further treats anxiety disorder-associated depression in the subject.

17. A method of treating depression, comprising administering to a subject in need thereof a therapeutically effective amount of the isolated Lactobacillus plantarum PDA6 strain according to claim 1.

18. The method of claim 15, wherein administering comprises oral, nasogastric, or rectal administration.

19. The method of claim 15, comprising administering the therapeutically effective amount in multiple doses of the isolated Lactobacillus plantarum PDA6 strain or the composition.

20. The method of claim 19, comprising administering the therapeutically effective amount daily for at least a week.

21. The method of claim 15, comprising administering the therapeutically effective amount until the intestine of the subject is detectably colonized with Lactobacillus plantarum PDA6.

22. The method of claim 15, wherein the therapeutically effective amount is a range of 10⁵ CFU to 10¹³ CFU per day.

23.-24. (canceled)
